(19) United States
(12) Plant Patent Application Publication
(10) Pub. No.: US 2012/0278959 P1
(43) Pub. Date: Nov. 1, 2012

(54) Hop plant named 'HBC 291'

(50) ;

(76) Inventors: Eugene G. Probasco, (US); Jason Perrault, (US)

(21) Appl. No.: 13/385,142

(22) Filed: Feb. 3, 2012

Related U.S. Application Data
(60) Provisional application No. 61/463,788, filed on Feb. 23, 2011.

(51) Int. Cl. A01H 5/00

(52) U.S. Cl. PLT/236

(57) ABSTRACT

A new hop plant named ‘HBC 291’ is disclosed. The cones of ‘HBC 291’ mature in mid September, and yield a crop of 2000 to 2400 pounds per acre. ‘HBC 291’ is used for its unique aromatic and flavor qualities, high alpha acid content and exceptional yield.

### CROSS-REFERENCE TO RELATED APPLICATIONS

This application claims priority of co-pending provisional application Ser. No. 61/462,788.

### STATEMENT REGARDING FEDERALLY SPONSORED RESEARCH OR DEVELOPMENT

None

### GENUS AND SPECIES

Humulus lupulus

### VARIETY DENOMINATION

HBC 291

### BACKGROUND OF THE INVENTION

‘HBC 291’ is a product of a controlled breeding program carried out by the inventors in the Yakima Valley of Washington state. ‘HBC 291’ was one of several seedlings resulting from a cross made in 2002 of female parent ‘Glacier’ (not patented) and male parent ‘HBC 9902(2) (not patented). A single plant of ‘HBC 291’ was selected in 2003, and in 2004 was expanded to 70 plants, which were planted in the area of Toppenish, Wash. The plants were observed and evaluated for several years, and in 2009 were expanded for further observation and evaluation in the Toppenish, Wash. area. A one acre test plot of ‘HBC 291’ was established in 2009. Throughout several generations of asexual propagation, ‘HBC 291’ has been observed to retain its distinctive characteristics and remain true to type.

### BRIEF DESCRIPTION OF THE PHOTOGRAPH

FIG. 1 illustrates a mature ‘HBC 291’ hop plant grown on a trellis;

FIG. 2. illustrates a hop cone of the ‘HBC 291’ hop plant; and

FIG. 3. illustrates a leaf of the ‘HBC 291’ hop plant.

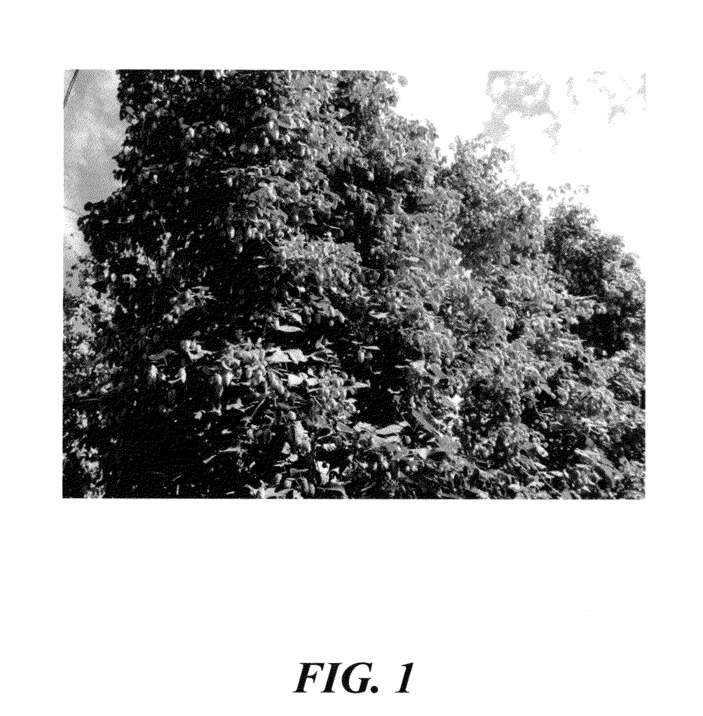

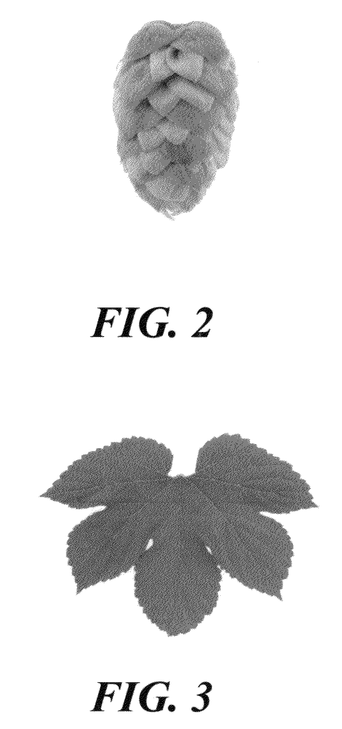

The colors of these illustrations may vary with lighting conditions and, therefore, color characteristics of this new variety should be determined with reference to the observations described herein, rather than from these illustrations alone.

### DETAILED BOTANICAL DESCRIPTION

The following description is based on observations made during the 2011 growing season at Toppenish, Wash. It should be understood that the characteristics described will vary somewhat depending upon cultural practices and climatic conditions, and can vary with location and season. Quantified measurements are expressed as an average of measurements taken from a number of individual plants of the new variety. The measurements of any individual plant or any group of plants, of the new variety may vary from the stated average.
Species: Humulus lupulus
   Ploidy.—Diploid.
   Use.—Brewing.
   Disease susceptibility.—‘HBC 291’ is not resistant to hop downy mildew. ‘HBC 291’ is resistant to powdery mildew.
   Pest susceptibility.—‘HBC 291’ is not resistant to normal infestations of hop aphid and normal infestations of two-spotted spider mite.
   Harvest date.—September 10-16 (2006-2009, and 2011 growing seasons, Toppenish, Wash.).
   Crop yield.—2000 to 2400 pounds per acre.
   Bine.—Color — Yellow green 145A Stipule direction — Reflexed Stipule color — Yellow green 144B Stripe — Absent Bine diameter — 10.0 mm at base; 9.0 mm at 9 feet; 8.0 mm at 18 feet.
   Leaf.—Arrangement — Opposite Shape — Simple Average length of mature leaf — 24.0 cm Average width of mature leaf — 19.0 cm Color of mature leaf upper surface — Yellow green 135A Color of mature leaf lower surface — Green 135A Color of immature leaf upper surface — Green 136A Color of immature leaf lower surface — Green 136B Number of lobes — 5 Margin — Lobed Serrate Serrations per inch — 4 Average petiole length (mature) — 9.0 cm Petiole color at base — Yellow green 145A Venation — Palmate Vein color — Yellow green 144C.
   Cone.—Average Weight — 1030 mg Average length — 3.5 cm Average diameter — 2.0 cm Bract tip color — Yellow green 143D Bract base color — Yellow green 143C Bracteole color — Yellow green 149D Cone shape — Oblong Bract shape — Ovate Bract tip shape — Cuspidate Bract tip position — Appressed Bracteole shape — Acute Lupulin Gland Color — Yellow green 154B Pickability — Good Cone storage — 30% loss of soft resins after 6 month storage at room temperature.
   Analytical characteristics.—Alpha acid 10.0% to 12.0% Beta acid 4.5% to 5.5% Cohumulone 21.0% to 23.0% Myrcene 37.0% to 39.0% Humulene 23.0% to 25.0% Caryophyllene 7.0% to 9.0% Farnesene 0.6% to 0.8% Linalool 1.0% to 1.5% Geraniol 0.8% to 1.1% Humulene/Caryophyllene Ratio 2.8% to 3.2% Total oil 1.5 to 2.5 mL per 100 g hops Storageability 70% to 72% alpha acids remaining after 6 months storage at room temperature.

What is claimed is:

1. A new and distinct hop plant as shown and described herein.
